(19) United States
(12) Patent Application Publication
(10) Pub. No.: US 2023/0181772 A1
(43) Pub. Date: Jun. 15, 2023

(54) ENGINEERED ANTI-PROSTATE STEM CELL ANTIGEN FUSION PROTEINS AND USES THEREOF

(71) Applicants: CITY OF HOPE, Duarte, CA (US); THE REGENTS OF THE UNIVERSITY OF CALIFORNIA, Oakland, CA (US)

(72) Inventors: Anna M. WU, Duarte, CA (US); Kirstin A. ZETTLITZ, Duarte, CA (US); Robert E. REITER, Los Angeles, CA (US)

(73) Assignees: CITY OF HOPE, Duarte, CA (US); THE REGENTS OF THE UNIVERSITY OF CALIFORNIA, Oakland, CA (US)

(21) Appl. No.: 17/999,352

(22) PCT Filed: May 18, 2021
(86) PCT No.: PCT/US2021/032970

§ 371 Date: Nov. 18, 2022

Related U.S. Application Data
(60) Provisional application No. 63/027,184, filed on May 19, 2020.

(51) Int. Cl. A61K 51/10; C07K 16/30

(52) U.S. Cl. CPC A61K 51/1093; A61K 51/1072; C07K 16/3069; A61K 2039/505

(57) ABSTRACT

Disclosed are engineered anti-prostate stem cell antigen (PSCA) fusion proteins and uses thereof to detect and treat cancers expressing PSCA.

### PRIORITY CLAIM

This application claims priority to U.S. Provisional Patent Application No. 63/027,184, filed May 19, 2020, the content of which is hereby incorporated by reference in its entirety.

### STATEMENT REGARDING FEDERALLY SPONSORED RESEARCH OR DEVELOPMENT

This invention was made with government support under Grant Number R01CA174294, awarded by the National Institutes of Health. The government has certain rights in the invention.

### SEQUENCE LISTING

This application contains a Sequence Listing, which was submitted in ASCII format via EFSWeb, and is hereby incorporated by reference in its entirety. The ASCII copy, created on May 18, 2021, is named SequenceListing.txt and is 11 KB in size.

### BACKGROUND

Human prostate stem cell antigen (PSCA) is a small glycosylphosphatidylinositol (GPO-anchored cell surface glycoprotein that is expressed in epithelial cells of prostate, urinary bladder, kidney, stomach and placenta. PSCA is overexpressed in prostate cancer and expression levels correlate with Gleason score and poor prognosis. PSCA is also highly expressed in prostate cancer metastases to lymph nodes and bone. PSCA expression is elevated in bladder cancer and pancreatic carcinoma. Previous work described the anti-human PSCA antibody 1G8 that specifically targets PSCA-expressing cells and inhibits tumor growth in preclinical models. The mouse IgG 1G8 was humanized by CDR-grafting (2B3) and affinity matured using yeast-display. Based on two affinity matured variants (A2 and A11) small, bivalent antibody fragment were engineered; the 80 kDa minibody (A11 Mb), a scFv-CH3 dimer and the cys-diabody (A2cDb), a 50 kDa scFv dimer functionalism with C-terminal cysteine residues. These antibody fragments display pharmacokinetics that are optimal for molecular imaging application such as short plasma half-life, and good tumor penetration. However, renal clearance prevents the use for radioimmunotherapy because of nephrotoxicity. Full-length IgGs exhibit clearance (other than target-mediated) mainly through the hepatobiliary route and the liver is less radiosensitive. Nevertheless, the long plasma half-life of IgGs (up to three weeks in humans) causes hematological toxicities when used in radioimmunotherapy. Therefore, antibodies having improved properties suitable for diagnostic and therapeutic uses need to be developed.

### SUMMARY

In one aspect, this disclosure relates to a genetically engineered anti-prostate stem cell antigen (PSCA) scFv-Fc fusion protein. The anti-PSCA scFv-Fc fusion protein comprises two peptides which form a homodimer, and each peptide comprises variable domains VH and VL of an anti-PSCA antibody, and a truncated hinge and fragment crystallizable (Fc) region. In some embodiments, the variable domains are arranged in the order of VH-VL. In some embodiments, the variable domains VH and VL are connected by a glycine-rich linker. In some embodiments, the linker is about 10-25 amino acids such as 10 amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids, 22 amino acids, 23 amino acids, 24 amino acids, or 25 amino acids. In some embodiments, the linker has a sequence of ((G4S)₂-GGSAQ) (SEQ ID NO: 1). In some embodiments, the FcRn binding region of the Fc region contains two point mutations H310A and H435Q. In some embodiments, the truncated hinge and Fc region is derived from human IgG2. In some embodiments, each peptide of the scFv-Fc fusion protein has an amino acid sequence at least 80%, at least 85%, at least 90%, at least 95%, at least 98%, or at least 99% identical to SEQ ID NO: 2 or SEQ ID NO: 3. In some embodiments, the scFv-Fc fusion protein is conjugated to an effector moiety including labeling moieties such as detectable markers including radioactive labels or fluorescent labels, and a therapeutic moiety. In certain embodiments, the therapeutic moiety includes a cytotoxic agent, an anti-tumor drug, a toxin, a radioactive agent, a cytokine, a second protein, an antibody, a radionuclide, or an enzyme.

In a related aspect, this disclosure relates to a pharmaceutical composition comprising an effective amount of one or more scFv-Fc fusion proteins, or scFv-Fc fusion protein and effector moiety conjugates disclosed herein. In some embodiments, the pharmaceutical composition further comprises one or more pharmaceutically acceptable carriers, excipients, and/or stabilizers. In some embodiments, the pharmaceutical composition further comprises one or more additional therapeutic agents including, for example, chemotherapeutic agents, cytotoxic agents, cytokines, growth inhibitory agents, radionuclides, and anti-hormonal agents. The therapeutic agent can be conjugated to the scFv-Fc fusion protein. In some embodiments, the pharmaceutical composition is formulated suitable for intravenous, intramuscular, intraperitoneal, intracerobrospinal, subcutaneous, intraarticular, intrasynovial, intrathecal, oral, topical, or inhalation administration.

In another aspect, disclosed is a method of treating or preventing a cancer expressing PSCA comprising administering to a subject an effective amount of one or more scFv-Fc fusion proteins, scFv-Fc fusion protein and effector moiety conjugates, or pharmaceutical compositions disclosed herein. In some embodiments, the method further entails administering to the subject one or more additional therapeutic agents including, for example, chemotherapeutic agents, cytotoxic agents, cytokines, growth inhibitory agents, radionuclides, and anti-hormonal agents. In some embodiments, the cancer expressing PSCA includes prostate cancer, pancreatic cancer, and bladder cancer.

In another aspect, disclosed herein is a method of detecting cancer expressing PSCA in a subject. The method entails administering one or more scFv-Fc fusion proteins disclosed herein to a subject, measuring the level of the scFv-Fc fusion proteins in the subject, comparing the level of the scFv-Fc fusion proteins with that of a healthy subject or with an average level of a healthy population, wherein an elevated level of the scFv-Fc fusion proteins in the subject indicating the presence of cancer. In some embodiments, the scFv-Fc fusion protein is conjugated with a labeling moiety. In some embodiments, the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable. In some embodiments, the cancer expressing PSCA includes prostate cancer, pancreatic cancer, and bladder cancer.

In another aspect, disclosed herein is a method of determining the prognosis of treating a cancer expressing PSCA in a subject. The method entails administering one or more scFv-Fc fusion proteins disclosed herein to a subject, measuring the level of the scFv-Fc fusion proteins in the subject, comparing the level of the scFv-Fc fusion proteins before and after the subject receives a cancer therapy, wherein a decreased level of the scFv-Fc fusion proteins in the subject after receiving the cancer therapy indicating that the cancer therapy is effective. In some embodiments, the scFv-Fc fusion protein is conjugated with a labeling moiety. In some embodiments, the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable. In some embodiments, the cancer expressing PSCA includes prostate cancer, pancreatic cancer, and bladder cancer.

In another aspect, disclosed herein is a method of imaging a cancer expressing PSCA in a subject. The method entails administering one or more scFv-Fc fusion proteins disclosed herein to a subject, and imaging the subject to determine the location and size of the tumor. In some embodiments, the scFv-Fc fusion protein is conjugated with a labeling moiety. In some embodiments, the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable. In some embodiments, the cancer expressing PSCA includes prostate cancer, pancreatic cancer, and bladder cancer.

### BRIEF DESCRIPTION OF THE DRAWINGS

This application contains at least one drawing executed in color. Copies of this application with color drawing(s) will be provided by the Office upon request and payment of the necessary fees.

FIG. 1 shows the design scheme of the gene encoding for the single-chain Fv-Fc2 fusion proteins and the resulting protein dimer. The left panel shows a scheme of the gene encoding for the single-chain Fv-Fc fusion protein. The sequence of the full-length human gamma 2 hinge is shown as SEQ ID NO: 5: VECPPCP APPVAGPS, where the lower hinge is encoded by CH2. The truncated residues (E)RKCC are shown with strike-through, and cysteine (C) residues that form disulfide bridges in the homodimerized protein are shown in bold and underlined. The strength of Fc-effector functions according to literature are depicted using + and −. The half-life of IgG2 is 21 days in human and 10-12 days in mice. The right panel shows a scheme of the scFv-Fc2 protein assembled to a homodimer. The blue dots represent the original murine CDRs that were crafted onto human sequences, which are depicted in red.

FIG. 2 shows the purified A2scFv-Fc2 and A2scFv-Fc2DM. SDS-PAGE analysis (2 μg/lane) under non-reducing and reducing conditions.

FIG. 3 shows size exclusion chromatography of purified A2scFv-Fc2 and A2scFv-Fc2DM (left panel) and interpolation of unknown molecular mass from the linear calibration curve (log MW/(VeN0)) (right panel).

FIG. 4 shows immunoblot with hPSCA-mFc (0.5 μg/lane, non-reducing and reducing). Immunoblots were probed with goat anti-mouse IgG-HRP to show presence of antigen (first panel). A2scFv-Fc2 (second panel) and A2scFv-Fc2DM (third panel) at 5 μg/mL followed by goat anti-human IgGFc-AP (Sigma-Aldrich 12136). Detection antibody only (fourth panel, goat anti-human IgGFc-AP). Blots were developed using BCIP/NBT (Millipore).

FIG. 5 shows binding of anti-PSCA scFv-Fc's to immobilized antigen hPSCA-mFc by ELISA. Binding of anti-PSCA scFv-Fc's was detected with goat anti-human IgGFc-AP antibody and developed using Alkaline Phosphatase Yellow (pNPP) liquid substrate (Sigma-Aldrich P7998).

FIG. 6 shows ELISA saturation binding curve of 1 of 3 independent experiments, duplicates were fitted using single-site specific binding model (GraphPad Prism 8).

FIG. 7 shows the sequence of SEQ ID NO: 2.

FIG. 8 shows the sequence of SEQ ID NO: 3.

FIGS. 9A-9C show flow cytometry analysis of binding of A2scFvFc2/DM to PSCA-expressing cell lines. FIG. 9A: No non-specific binding of anti-PSCA antibody fragments was observed, while strong binding to prostate cancer cell line 22Rv1-PSCA indicated antigen specificity. FIG. 9B: Titration of antibody binding to constant cell numbers was used to determine the apparent on-cell affinity (half-maximal binding). FIG. 9C: A2scFv-Fc2 was used to confirm PSCA-expression of transduced murine cell lines RM9 and KPC, as well as endogenous PSCA expression of the human Capan-1.

FIGS. 10A-10D show radiolabeling of A2scFvFc2/DM. FIG. 10A: SDS-PAGE of DFO-conjugated and unconjugated antibody fragments under non-reducing and reducing conditions (Coomassie stained). FIG. 10B: SEC elution profiles of unconjugated and DFO-conjugated A2scFvFc2/DM. FIG. 10C: Scheme of radiolabeling procedure and radiolabeling results. FIG. 10D: SEC of unconjugated and ⁸⁹Zr-DFO-conjugated antibody fragments.

FIGS. 11A-11B show ex vivo biodistribution of ⁸⁹Zr-A2scFvFc2/DM in nude mice. FIG. 11A: Ex vivo biodistribution at 4, 24 and 96 hours p.i., depicted as box-and-whisker (min-to-max). Asterisks indicate significance as analyzed by 2-way ANOVA, Tukey (multiple comparison test) corrected. FIG. 11B: Ex vivo biodistribution values (mean±SEM) and resulting ratios in clearance tissues.

FIG. 12 shows in vivo biodistribution and tumor targeting. ImmunoPET imaging of nude mice (22Rv1-PSCA, right shoulder) injected with ⁸⁹Zr-A2scFv-Fc2DM (upper panel) or ⁸⁹Zr-A2scFv-Fc2 (lower panel). Depicted are representative scans as whole-body maximum-intensity projection PET/CT overlays.

FIG. 13 shows immunoPET in a syngeneic prostate cancer model in hPSCA KI mice. ⁸⁹Zr-A2scFv-Fc2 or ⁸⁹Zr-A2scFv-Fc2DM (10 μg/70 μCi) was injected into hPSCA KI mice bearing bilateral PSCA−/+RM9 subcutaneous tumors (left panel) or unilateral RM9-PSCA s.c. tumors (right panel). The mice were imaged at 4, 24 and 96 hours p.i. Depicted are representative scans as whole-body maximum-intensity projection PET/CT overlays.

FIGS. 14A-14B show ex vivo biodistribution of ⁸⁹Zr-A2scFvFc2/DM in hOPSCA KI mice. FIG. 14A: Ex vivo biodistribution at 24 and 96 hours p.i., depicted as box-and-whisker (min-to-max). Asterisks indicate significance as analyzed by 2-way ANOVA, Tukey (multiple comparison test) corrected. FIG. 14B: Ex vivo biodistribution values (mean±SEM) and resulting ratios in clearance tissues.

FIGS. 15A-15B show terminal half-lives of ⁸⁹Zr-A2scFv-Fc2/DM in tumor-bearing nude mice.

### DETAILED DESCRIPTION

scFv-Fc Fusion Protein

Disclosed herein is a genetically engineered anti-prostate stem cell antigen (PSCA) scFv-Fc fusion protein. In various embodiments, the anti-PSCA scFv-Fc fusion protein comprises two peptides which form a homodimer, and each peptide comprises variable domains VH and VL of an anti-PSCA antibody, arranged in the order of VH-VL, engineered human IgG2 Fc domains, with reduced effector functions (such as low antibody-dependent cellular cytotoxicity (ADCC) and complement-dependent cytotoxicity (CDC)), and point mutations such as H310A and H435Q in the FcRn binding region of the Fc region for rapid clearance from circulation after administrating to a subject. The variable domains are connected by a glycine-rich linker to form an scFv.

The term “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

In some embodiments, the scFv-Fc fusion protein disclosed herein comprises variable domains derived from anti-PSCA antibody A2, which is disclosed in U.S. Pat. No. 9,527,919, the content of which is incorporate by reference in its entirety and particularly with reference to its disclosure of antibody and antibody fragments such as VH, VL and CDR sequences, the structure of scFv and scFv-Fc, methods of making them, and their pharmaceutical compositions and formulations.

In some embodiments, the VH and VL domains of the scFv-Fc fusion protein disclosed herein have sequences substantially identical to those of the VH and VL domains of the A2 antibody disclosed in U.S. Pat. No. 9,527,919.

The term “substantially identical” in the context of two or more nucleic acid or amino acid sequences, refers to two or more sequences or subsequences that are the same or have a specified percentage of nucleotides or amino acid residues that are the same (i.e., about 80% identity, preferably at least 65%, 70%, 75%, 80%, 85%, 90%, 91%, 92%, 93%, 94%, 95%, 96%, 97%, 98%, 99%, or higher identity) over the referenced sequences or portions, when compared and aligned for maximum correspondence over a comparison window or designated region) as measured using a BLAST or BLAST 2.0 sequence comparison algorithms, or by manual alignment and visual inspection (see, e.g., NCBI web site or the like). The definition also includes sequences that have deletions and/or additions, as well as substitutions. Preferably, the sequence identity is at least 85%, 90%, 95%, 97%, 98% or 99% between two referenced domains. In some embodiments, the difference in sequence is just by one, two, three or four, or from five to 12, amino acids as to referenced sequence or domain. As described below, the preferred algorithms can account for gaps and the like. Preferably, identity exists over a region that is at least about 15 amino acids or nucleotides in length, or more preferably over a region that is 15-50 amino acids or nucleotides in length. In other embodiments, the identity may exist over a region that is at least about 50, 100, 150, 200, or more amino acids.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of this disclosure.

The following eight groups each contain amino acids that are conservative substitutions for one another: 1) Alanine (A), Glycine (G); 2) Aspartic acid (D), Glutamic acid (E); 3) Asparagine (N). Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

In some embodiments, the scFv-Fc fusion protein is A2scFv-Fc2, VH-linker-VL-(hg2)hinge-CH2-CH3, each peptide has a sequence represented by SEQ ID NO: 2:

EVQLVESGGGLVQPGGSLRLSCAASGFNIKDYYIHWVRQAPGKGLEWVAWIDPEYGDSEFVPKFQ
<------------------------------VH--------------------------------
GRATMSADTSKNTAYLQMNSLRAEDTAVYYCKTGGFWGRGTLVTVSSGGGGSGGGGSGGSAQD
------------VH--------------------------------><---linker----><
IQLTQSPSSLSASVGDRVTITCSASSSVRFIHWYQQKPGKAPKRLIYDTSKLASGVPSRFSGSGSG
-------------------------VL---------------------------------------
TDFTLTISSLQPEDFATYYCQQWGSSPFTFGQGTKVEIKVECPPCPAPPVAGPSVFLFPPKPKDTL
-----------------VL-------------------><-hinge-><-------------CH2-
MISRTPEVTCVVVVDVSHEDPEVQFNWYVDGMEVHNAKTKPREEQFNSTFRVVSVLTVVHQDWLN
---------------CH2-----------------------------------------------
GKEYKCKVSNKGLPAPIEKTISKTKGQPREPQVYTLPPSREEMTKNQVSLTQLVKGFYPSDIAVEW
---CH2------------------><----------------------CH3---------------
ESNGQPENNYKTTPPMLDSDGSFFLYSKLTVDKSRWQQGNVFSQSVMHEALHNHYTQKSLSLSP
------------------------------CH3-------------------------------
GK
->

In some embodiments, the scFv-Fc fusion protein is A2scFv-Fc2DM (with double mutations H310A/H435Q), VH-linker-VL-(hg2)hinge-CH2-CH3DM, each peptide has a sequence represented by SEQ ID NO: 3:

EVQLVESGGGLVQPGGSLRLSCAASGFNIKDYYIHWVRQAPGKGLEWVAWIDPEYGDSEFVPKFQ
<------------------------------VH--------------------------------
GRATMSADTSKNTAYLQMNSLRAEDTAVYYCKTGGFWGRGTLVTVSSGGGGSGGGGSGGSAQD
------------VH--------------------------------><---linker----><
IQLTQSPSSLSASVGDRVTITCSASSSVRFIHWYQQKPGKAPKRLIYDTSKLASGVPSRFSGSGSG
-------------------------VL---------------------------------------
TDFTLTISSLQPEDFATYYCQQWGSSPFTFGQGTKVEIKVECPPCPAPPVAGPSVFLFPPKPKDTL
-----------------VL-------------------><-hinge-><-------------CH2-
MISRTPEVTCVVVDVSHEDPEVQFNWYVDGMEVHNAKTKPREEQFNSTFRVVSVLTVVAQDWLN
---------------CH2-----------------------------------------------
GKEYKCKVSNKGLPAPIEKTISKTKGQPREPQVYTLPPSREEMTKNGVSLTCLVKGFYPSDIAVEW
---CH2------------------><----------------------CH3---------------
ESNGQPENNYKTTPPMLDSDGSFFLYSKLTVDKSRWQQGNVFSCSVMHEALHNQYTQKSLSLSP
------------------------------CH3-------------------------------
GK
->

In some embodiments, the antibody fragments and/or domains of the disclosed fusion protein are modified, i.e., by the covalent attachment of any type of molecule to the antibody fragments and/or domains. For example, the modifications include, without limitation, glycosylation, acetylation, pegylation, phosphorylation, amidation, derivatization by known protecting/blocking groups, proteolytic cleavage, linkage to a cellular ligand or other protein, and the like. Any of numerous chemical modifications may be carried out by known techniques, including, but not limited to specific chemical cleavage, acetylation, formylation, metabolic synthesis of tunicamycin, etc. Additionally, the modifications include one or more non-natural amino acids.

The disclosed engineered fusion proteins recognize and specifically bind to PSCA with high affinity. These genetically engineered fusion proteins are tailored specifically for in vivo use for targeting and detection various cancers expressing PSCA, such as prostate cancer, pancreatic cancer and bladder cancer.

The phrase “specific binding” means that an engineered fusion protein binds to a particular target protein such as PSCA at least two times the background and more typically more than 10 to 100 times background. A variety of immunoassay formats may be used to select fusion proteins that specifically bind to PSCA. For example, solid-phase ELISA immunoassays are routinely used to select antibodies specifically binding to a target protein (see, e.g., Harlow & Lane, Using Antibodies, A Laboratory Manual (1998) for a description of immunoassay formats and conditions that can be used to determine specific immunoreactivity).

PSCA and its expression in cancer of the prostate, bladder, and pancreas is disclosed in U.S. Pat. No. 6,756,036 which is incorporated by reference in its entirety. The human PSCA translated amino acid sequence is represented by SEQ ID NO: 4 (UniProtKB-O43653), where the signal peptide is shown underlined and the propeptide is shown in bold and italic:

MAGLALQPGTALLCYSCKAQVSNEDCLQVENCTQLGEQCWTA
RIRAVGLLTVISKGCSLNCVDDSQDYYVGKKNITCCDTDLCN
A

[symbol]

[symbol]

[symbol]

[symbol]

Also disclosed are polynucleotides comprising a nucleotide sequence encoding an antibody of the invention and fragments thereof. In certain embodiments, this disclosure provides an expression vector encoding the fusion protein disclosed herein. In certain embodiments, this disclosure provides polynucleotides encoding the fusion protein disclosed herein for use in gene therapy or in vivo administration.

In certain embodiments, the fusion protein disclosed herein is conjugated to an “effector” moiety. The effector moiety can be various molecules, including labeling moieties such as detectable markers including radioactive labels or fluorescent labels, or can be a therapeutic moiety. Such effector moieties include but are not limited to, a cytotoxic agent, an anti-tumor drug, a toxin, a radioactive agent, a cytokine, a second protein, an antibody, or an enzyme. Further, the fusion protein disclosed herein can be linked to an enzyme that converts a prodrug into a cytotoxic agent.

Examples of cytotoxic agents include, but are not limited to ricin, doxorubicin, daunorubicin, TAXOL, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, and glucocorticoid and other chemotherapeutic agents, as well as radioisotopes. Suitable detectable markers include, but are not limited to, a radioisotope, a fluorescent compound, a bioluminescent compound, chemiluminescent compound, a metal chelator or an enzyme. The second protein can include, but is not limited to, an enzyme, lymphokine, oncostatin or toxin. Suitable toxins include doxorubicin, daunorubicin, TAXOL, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, ricin, abrin, glucocorticoid and radioisotopes.

Techniques for conjugating therapeutic agents to constructs according to the invention are well known (see, e.g., Amon et al., “Monoclonal Antibodies For Immunotargeting Of Drugs. In Cancer Therapy,” in Monoclonal Antibodies And Cancer Therapy, Reisfeld et al. (eds.), pp. 243-56 (Alan R. Liss, Inc. 1985) Hellstrom et al., “Antibodies For Drug Delivery’ in Controlled Drug Delivery (2nd Ed.), Robinson et al. (eds.), pp. 623-53 (Marcel Dekker, Inc. 1987); Thorpe, “Antibody Carriers Of Cytotoxic Agents. In Cancer Therapy: A Review” in Monoclonal Antibodies 84: Biological And Clinical, Applications, Pinchera et al. (eds.), pp. 475-506 (1985) and Thorpe et al., “The Preparation And Cytotoxic Properties Of Antibody-Toxin Conjugates. Immunol. Rev., 62: 119-58 (1982)).

### Pharmaceutical Compositions

Also disclosed is a pharmaceutical composition comprising an effective amount of one or more anti-PSCA scFv-Fc fusion proteins or one or more anti-PSCA scFv-Fc fusion protein and effector moiety conjugates disclosed herein. The pharmaceutical composition can further comprise one or more additional therapeutic agents, and/or one or more pharmaceutically acceptable carriers, excipients, and stabilizers.

Acceptable carriers, excipients or stabilizers can be acetate, phosphate, citrate, and other organic acids; antioxidants (e.g., ascorbic acid) preservatives low molecular weight polypeptides; proteins, such as serum albumin or gelatin, or hydrophilic polymers such as polyvinylpyllolidone; and amino acids, monosaccharides, disaccharides, and other carbohydrates including glucose, mannose, or dextrins; chelating agents; and ionic and non-ionic surfactants (e.g., polysorbate); salt-forming counter-ions such as sodium; metal complexes (e.g. Zn-protein complexes); and/or non-ionic surfactants. The fusion protein, or the conjugate can be formulated at a concentration of between 0.5-200 mg/ml, or between 10-50 mg/ml.

Additional therapeutic agents include, for example, chemotherapeutic agents, cytotoxic agents, cytokines, growth inhibitory agents, anti-hormonal agents, and radionuclide including alpha- or beta-emitting radioisotopes such as At-211, Ac-225, Cu-67, Y-90, Lu-177, and 1-131. The therapeutic agents may also be prepared as sustained-release preparations (e.g., semi-permeable matrices of solid hydrophobic polymers (e.g., polyesters, hydrogels (for example, poly (2-hydroxyethyl-methacrylate), or poly (vinylalcohol)), polylactides. The fusion proteins, or conjugates may also be entrapped in microcapsules prepared, for example, by coacervation techniques or by interfacial polymerization, for example, hydroxymethylcellulose or gelatin microcapsules and poly-(methylmethacy late) microcapsules, respectively, in colloidal drug delivery systems (for example, liposomes, albumin microspheres, microemulsions, nano-particles and nanocapsules) or in macroemulsions. In certain embodiments, the therapeutic agent is conjugated to the anti-PSCA scFv-Fc fusion protein.

The term “effective amount” “therapeutically effective amount” or “therapeutically effective dose” as used herein refers to an amount of a composition that produces a desired effect. An effective amount of a fusion protein, a conjugate, or a pharmaceutical composition may be used to produce a therapeutic effect in a subject, such as preventing or treating a target condition, alleviating symptoms associated with the condition, or producing a desired physiological effect. In such a case, the effective amount is a “therapeutically effective amount,” “therapeutically effective concentration” or “therapeutically effective dose.” The precise effective amount or therapeutically effective amount is an amount of the fusion protein, the conjugate, or the pharmaceutical composition that will yield the most effective results in terms of efficacy of treatment in a given subject. This amount will vary depending upon a variety of factors, including but not limited to the characteristics of the active agent (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. Further, an effective or therapeutically effective amount may vary depending on whether the fusion protein, the conjugate, or the pharmaceutical composition is administered alone or in combination with another composition, drug, therapy or other therapeutic method or modality. One skilled in the clinical and pharmacological arts will be able to determine an effective amount or therapeutically effective amount through routine experimentation, namely by monitoring the subject's response to administration of the fusion protein, the conjugate, or the pharmaceutical composition and adjusting the dosage accordingly. For additional guidance, see Remington: The Science and Practice of Pharmacy, 21st Edition, Univ. of Sciences in Philadelphia (USIP), Lippincott Williams & Wilkins, Philadelphia, Pa., 2005, which is hereby incorporated by reference as if fully set forth herein.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The pharmaceutical composition can, if desired, also contain other compatible therapeutic agents.

Preferred pharmaceutical preparations deliver one or more fusion proteins, or conjugates disclosed herein, optionally in combination with one or more chemotherapeutic agents or immunotherapeutic agents, in a sustained release formulation. The fusion proteins, or conjugates disclosed herein may be administered therapeutically as a sensitizing agent that increases the susceptibility of tumor cells to other cytotoxic cancer therapies, including chemotherapy, radiation therapy, immunotherapy and hormonal therapy.

Use of the scFv-Fc Fusion Protein

The scFv-Fc fusion proteins disclosed herein can be used in the diagnosis, prognosis and treatment of cancers which overexpress PSCA, for example, prostate, pancreatic and bladder cancers. Thus, this disclosure also relates to a method of diagnosing, prognosing, or treating a cancer overexpressing PSCA by administering an effective amount of the scFv-Fc fusion proteins or the pharmaceutical compositions disclosed herein to a subject. In certain embodiments the method is applied to hormone refractory or therapy resistant cancers. In certain embodiments the method is applied to metastatic cancers.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. Treatment may also mean a prophylactic or preventative treatment of a condition.

The fusion proteins, conjugates and pharmaceutical compositions disclosed herein can be administered for therapeutic or prophylactic treatments. In therapeutic applications, compositions are administered to a patient suffering from a disease (e.g., cancer) in a “therapeutically effective dose.” Amounts effective for this use will depend upon the severity of the disease and the general state of the patient's health. Single or multiple administrations may be administered depending on the dosage and frequency as required and tolerated by the patient. A “patient’ or “subject’ for the purposes of the present invention includes both humans and other animals, particularly mammals. Thus, the methods are applicable to both human therapy and veterinary applications. Other known cancer therapies can be used in combination with the methods of the invention. For example, the fusion protein, the conjugate, or the pharmaceutical composition for use according to this disclosure may also be used to target or sensitize a cell to other cancer therapeutic agents such as 5FU, vinblastine, actinomycin D, cisplatin, methotrexate, and the like.

In other embodiments, the methods may be practiced together with other cancer therapies (e.g., radical prostatectomy), radiation therapy (external beam or brachytherapy), hormone therapy (e.g., orchiectomy, LHRH-analog therapy to suppress testosterone production, anti-androgen therapy), or chemotherapy. Radical prostatectomy involves removal of the entire prostate gland plus some surrounding tissue. This treatment is used commonly when the cancer is thought not to have spread beyond the tissue. Radiation therapy is commonly used to treat prostate cancer that is still confined to the prostate gland, or has spread to nearby tissue. If the disease is more advanced, radiation may be used to reduce the size of the tumor. Hormone therapy is often used for patients whose prostate cancer has spread beyond the prostate or has recurred. The objective of hormone therapy is to lower levels of the male hormones, androgens and thereby cause the prostate cancer to shrink or grow more slowly. Luteinizing hormone-releasing hormone (LHRH) agonists decrease the production of testosterone. These agents may be injected either monthly or longer. Two such analogs are leuprolide and Goserelin. Anti-androgens (e.g., flutamide, bicalutamide, and nilutamide) may also be used. Total androgen blockade refers to the use of anti-androgens in combination with orchiectomy or LHRH analogs. Chemotherapy is an option for patients whose prostate cancer has spread outside of the prostate gland and for whom hormone therapy has failed. It is not expected to destroy all of the cancer cells, but it may slow tumor growth and reduce pain. Some of the chemotherapy drugs used in treating prostate cancer that has returned or continued to grow and spread after treatment with hormonal therapy include doxorubicin (Adriamycin), estramustine, etoposide, mitoxantrone, vinblastine, and paclitaxel. Two or more drugs are often given together to reduce the likelihood of the cancer cells becoming resistant to chemotherapy. Small cell carcinoma is a rare type of prostate cancer that is more likely to respond to chemotherapy than to hormonal therapy.

The combined administrations contemplate co-administration, using separate formulations or a single pharmaceutical formulation, and consecutive administration in either order, wherein preferably there is a time period while both (or all) active agents simultaneously exert their biological activities.

Treatment will generally involve the repeated administration of the fusion proteins or the pharmaceutical compositions via an acceptable route of administration such as intravenous injection (IN), at an effective dose. Dosages will depend upon various factors generally appreciated by those of skill in the art, including without limitation, the type of cancer and the severity, grade, or stage of the cancer, the binding affinity and half-life of the fusion proteins used, the desired steady-state concentration level, frequency of treatment, and the influence of chemotherapeutic agents or other therapeutic agents used in combination with the treatment method of the invention. Typical doses may range from about 0.1 to 100 mg/kg. Doses in the range of 10-500 mg of the fusion proteins per week may be effective and well tolerated, although even higher weekly doses may be appropriate and/or well tolerated. In certain embodiments, the administration schedule is weekly or every 2-4 weeks. The principal determining factor in defining the appropriate dose is the amount of a particular agent necessary to be therapeutically effective in a particular context. Repeated administrations may be required in order to achieve tumor inhibition or regression. Initial loading doses may be higher. The initial loading dose may be administered as an infusion. Periodic maintenance doses may be administered similarly, provided the initial dose is well tolerated.

In therapeutic use for the treatment of cancer, the dosage of the fusion proteins, or the conjugates may be varied depending upon the requirements of the patient, the severity of the condition being treated, and the active agent being employed. For example, dosages can be empirically determined considering the type and stage of cancer diagnosed in a particular patient. The dose administered to a patient should be sufficient to affect a beneficial therapeutic response in the patient over time. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

Direct administration of the fusion proteins is also possible and may have advantages in certain contexts. For example, for the treatment of bladder carcinoma, the fusion proteins or the pharmaceutical compositions may be injected directly into the bladder.

The fusion proteins, conjugates and pharmaceutical compositions disclosed herein can be administered to a subject using various known methods, such as intravenous administration, e.g., as a bolus or by continuous infusion over a period of time, by intramuscular, intraperitoneal, intracerobrospinal, subcutaneous, intraarticular, intrasynovial, intrathecal, oral, topical, or inhalation routes. Intravenous or subcutaneous administration is preferred. The administration may be local or systemic.

The pharmaceutical compositions for administration will commonly comprise a fusion protein, or a conjugate as described herein dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patients' needs.

The pharmaceutical compositions can be administered in a variety of dosage forms depending upon the method of administration. For example, unit dosage forms suitable for oral administration include, but are not limited to, powder, tablets, pills, capsules and lozenges. It is recognized that when administered orally, the pharmaceutical composition should be protected from digestion. This is typically accomplished either by complexing the active agent with a composition to render them resistant to acidic and enzymatic hydrolysis, or by packaging the active agents in an appropriately resistant carrier, such as a liposome or a protection barrier. Means of protecting agents from digestion are well known in the art.

Methods of Detecting Cancer and/or Tumor Imaging

In another aspect, disclosed herein is a method of detecting cancer or tumor imaging in vivo through administration of the anti-PSCA scFv-Fc fusion protein disclosed herein. In one embodiment, provided herein is a method of imaging a cancer cell in vivo, the method comprising administering a labeled anti-PSCA scFv-Fc fusion protein to a mammal and imaging the fusion protein in vivo. The mammal, includes without limitation, a mouse, rat, hamster, rabbit, pig, human, and the like.

A “label” or a “detectable moiety” or “detectable marker” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

Methods of in vivo imaging are well known in the art and include without limitation, magnetic resonance imaging (MRI), nuclear magnetic resonance (NMR) (R. Weissleder, 1999, Radiology 212:609-14), computerized axial tomography (CAT) scan, cooled charged coupled device (CCD) camera optical imaging (Honigman, et al., 2001 Mol. Ther. 4:239-249), bioluminescent optical imaging (P R Contag, et al., 1998 Nat. Med. 4:245-247), position emission tomography (PET) (ME Phelps, 1991 Neurochemical Research 16:929-994: J G Tuvajev, et al., 1998 Cancer Res 58:4333-4341), single photon emission computed tomography (J G. Tuvajev, et al., 1996 Cancer Res. 45:4087-4095), microPET (reviewed in McVeigh, 2006, Circ. Res. 98:879-86), and the like.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Materials and Methods

Design and cloning of scFv-Fc fragments: The single-chain variable fragment-crystallizable fragment fusion protein (scFv-Fc) consists of the scFv fused to the Fc region of human IgG2. The scFv in VH-VL sequence is connected by a 15 amino acid glycine-rich linker (G₄S)₂-GGSAQ. The human gamma 2 Fc domain contains a truncated hinge followed by CH2 and CH3 domains. The resulting fusion protein is named scFv-Fc2 forms. Point mutations H310A and H435Q in the FcRn binding region of the Fc were introduced for construct scFv-Fc2DM (double mutant). Both wildtype and mutated scFv-Fc peptides can form dimers of approximately 110 kDa.

The synthetic codon-optimized DNA (in plasmid pMA-T, Invitrogen by ThermoFisher Scientific) encoding for the two A2scFv-Fc constructs were sub-cloned into the mammalian expression vector pSecTag2A(AgeI) using the restriction sites AgeI and ApaI The resulting plasmids were transfected into mammalian cell line FS293-F (FreeStyle™ 293-F cells (Gibco, ThermoFisher Scientific) using Lipofectamine™ 3000 (ThermoFisher Scientific). Stable cell pools were generated by Zeocin™ Selection Reagent (ThermoFisher Scientific) selection and tested for protein expression by Western blot.

Protein production: Zeocin selected stable cell pools were expanded into Nunc TripleFlask and growth medium was replaced with serum-free medium (Opti-MEM®, ThermoFisher Scientific) for protein expression. Cell culture supernatant was collected every 3-4 days for two weeks.

Protein purification: Recombinant anti-PSCA scFv-Fc2 fusion proteins were purified from cell culture supernatants using an Äkta™ chromatography system (GE Healthcare) with columns HiTrap® rProtein A FF for A2scDv-Fc2 and HiTrap® Protein L for A2scFv-FcDM (both GE Healthcare). Columns were equilibrated using 5 column volumes of binding buffer (20 mM sodium phosphate pH 7.0). Prior to loading supernatants were concentrated and adjusted to pH 8.0-9.0 by adding 1/10 volume of 1M TrisHCl pH 9.0. Unbound protein was removed by washing the column with binding buffer. Bound protein was eluted with 100 mM citric acid pH 3.0, neutralized by adding 1/10 volume 1M TrisHCl pH 9.0 and protein containing fractions were dialyzed against PBS.

Biochemical characterization: Purified proteins were analyzed by SDS-PAGE, Western blot and size exclusion chromatography (Superdex®200 Increase 10/300 GL, ThermoFisher Scientific) for purity, integrity and apparent molecular weight.

Western Blot was also used to test antigen specificity. Recombinant human PSCA mouse Fc fusion protein (UCLA) was blotted and anti-PSCA scFv-Fc variants were tested for binding. Specificity and affinity of antigen binding was further evaluated by performing saturation binding on immobilized antigen in ELISA. Specific binding was fitted using the “one site-specific binding” equation to determine the Kd (ligand concentration that binds half the antigen sites at equilibrium) and Bmax (maximum number of binding sites).

Cell lines and mouse models: Human prostate cancer cell line 22Rv1, mouse prostate cancer cell line RM9 (ffluc) and mouse pancreatic cancer cell line KPC (ffluc) and their human PSCA expressing derivatives (22Rv1-hPSCA, RM9-hPSCA (ffluc) and KPC-hPSCA (ffluc) (generated by retroviral gene transfer, G418 selection and flow cytometry fluorescence activated cell sorting) were cultured in RPMI 1640 (22Rv1) or DMEM supplemented with 10% FBS. RM9 and KPC cell lines were provided by the University of Texas MD Anderson Cancer Center and Dr. Saul Priceman (City of Hope). The human pancreatic cancer cell line Capan-1 (ATCC®HTB-79™) was cultured in IMDM, 20% FBS.

For the human prostate cancer xenograft model, male athymic nude mice (J/Nu, Jax 002019, 8-12 weeks) were implanted with 1-2×10⁶ cells (22Rv1-PSCA or 22Rv1) in 100 μL (1:1 vol:vol HBSS: Matrigel) subcutaneously in the shoulder area. Tumors were allowed to grow for 10-14 days. The human PSCA knock in (hPSCA KI) mouse model was generated by targeted insertion of hPSCA cDNA through homologous recombination in murine embryonic stem cells by standard gene-targeting methods and backcrossed onto C57BL6/J. The syngeneic murine prostate cancer model expressing human PSCA was generated by implanting 5×10⁴ cells (RM9 or RM9-hPSCA) in 100 μL (1:1 HBSS:Matrigel) into C57BL/6×hPSCA knock in mice (female, heterozygous) subcutaneously in the shoulder region and tumors were allowed to grow for 7-10 days. Some mice were implanted with bilateral PSCA-positive and negative tumors, respectively.

Cell binding and flow cytometry. Binding of the anti-PSCA A2scFv-Fc fusion proteins to cell lines transduced to express cell surface human PSCA or expressing endogenous hPSCA was analyzed by flow cytometry. Cells (0.5×10⁶) were incubated with A2scFvFc2 or A2scFvFc2DM (1 μM) in 0.5 mL PBA buffer (PBS 1×, 2% FBS, 0.02% sodium azide (NaN3) for 2 hours at 4° C. Cells were washed with 0.5 mL PBA twice and bound scFv-Fc was detected using goat anti-human IgG (H+L)-Alexa Fluor 647 secondary antibody (1:1000 dilution) for 30 minutes at 4° C. Cells were analyzed on a BD LSRFortessa™ X-20 Flow Cytometer (BD Biosciences) and displayed using FlowJo.

Conjugation with bifunctional chelator. Anti-PSCA A2scFv-Fc2 and A2scFv-Fc2DM were incubated with p-SCN-Bn-Deferoxamine (SCN-DFO, Macrocyclics, B-705) at 5-fold molar excess (ph 9.0, 2 hours, room temperature). Excess SCN-DFO was removed using Micro Bio-Spin chromatography columns (BioRad) preconditioned with PBS. Successful conjugation was confirmed by SDS-PAGE (Coomassie-stained) and SEC (Superdex 200) analysis.

Radiolabeling: [⁸⁹Zr]Zr-oxalate (3D Imaging LLC) was neutralized (0.45 volume 2 M Na₂CO₃, 2.5 volume 1M HEPES) and added to the DFO-conjugated protein (0.185-0.278 MBq/5-7.5 μCi/μg) for 1 hour at room temperature. Radiolabeled protein was purified using Micro Bio-Spin chromatography columns (BioRad). Labeling efficiency and radiochemical purity were determined by ITLC using 20 mM citric acid (pH 5.0) as solvent.

ImmunoPET/CT (in vivo): Mice were injected via the tail vein with 10 μg (1.3-2.6 MBq/35-70 μCi) of ⁸⁹Zr-A2scFvFc2 or ⁸⁹Zr-A2scFvFc2DM. Mice were anesthetized with 2-3% isoflurane and 10-minute static PET scans followed by a 1-minute standard CT scan were acquired on the GNEXT PET/CT scanner (Sofie Biosciences) at indicated time points post injection (p.i.). Images were reconstructed using an 3D-OSEM MAP algorithm and are presented as whole-body maximum intensity projection (MIP) PET/CT overlays using AMIDE software.

Biodistribution (ex vivo): Tissues of interest were dissected, weighed and gamma counted (2480 Wizard2 Gamma counter, Perkin Elmer). Percent injected dose per gram of tissue (% ID/g) was calculated based on decay-corrected injected dose standards.

Plasma Half-life: Blood samples (5 μL) were taken from the tail vein between 3 minutes and 96 hours and were gamma counted. Terminal half-lives (t_{1/2β}) were calculated using a two-phase decay model (GraphPad Prism 9).

### Example 1: Generation of A2scFv-Fc2 and A2scFv-Fc2DM

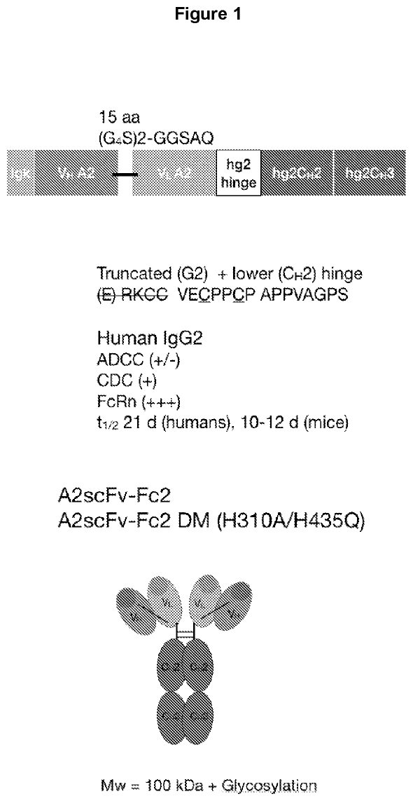

As illustrated in FIG. 1, Novel scFv-Fc fragments based on anti-PSCA antibody fragment A2 were designed by changing the order of the variable domains to VH-VL, connected by a 15 amino acid glycine-rich linker ((G45)₂-GGSAQ) followed by a human immunoglobulin 2 (IgG2) truncated hinge and fragment crystallizable (Fc). The double mutant (DM) derivative contains two point mutations replacing histidine residues involved in FcRn binding with alanine or glutamine, respectively (H310A/H435Q).

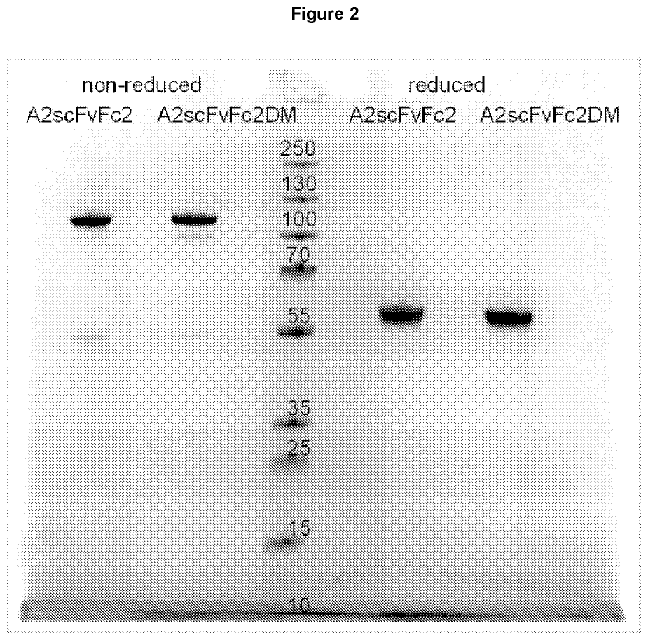

Codon optimized genes encoding the scFv-Fc proteins were subcloned into mammalian expression vector (pSECTag2A), transfected into FreeStyle 293-F cells and stable cell pools selected. Recombinant scFv-Fc fusion proteins were purified from mammalian cell culture supernatant. SDS-Page analysis of the purified protein under non-reducing conditions showed a single band with an apparent molecular mass of approximately 110 kDa (calculated MW 99.65 kDa for A2scFv-Fc2 and 99.5 kDa for A2scFv-Fc2DM) consistent with the glycosylated (Asn-297, CH2) homodimer (FIG. 2). Under reducing conditions the disulfide bridges in the hinge are broken and the monomeric protein migrates at approximately 55 kDa.

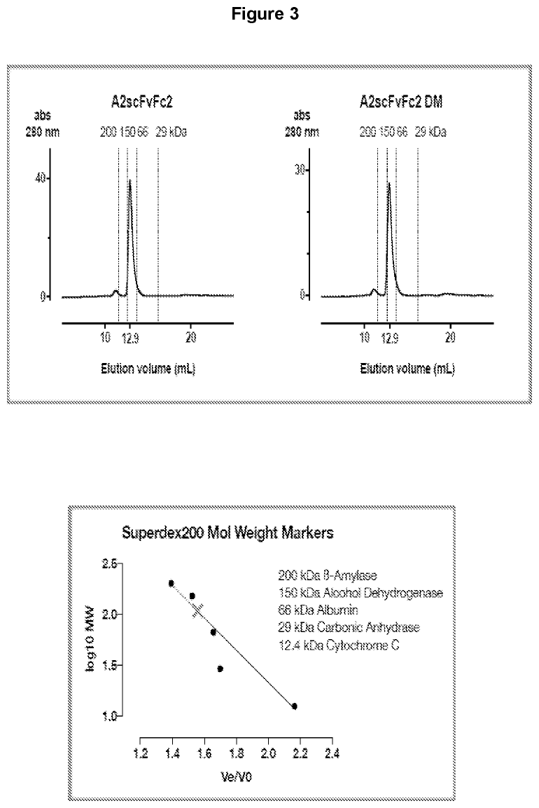

Purity, integrity and apparent molecular weight of the purified anti-PSCA scFv-Fc's were confirmed by size exclusion chromatography (FIG. 3). Both anti-PSCA scFv-Fc's eluted as single peaks (93-95% AUC) at elution volumes (12.92 and 12.88 mL, respectively) concordant with the calculated molecular weight of approximately 110 kDa. The molecular mass determination using Gel filtration molecular weight markers (Sigma-Aldrich, MWGF200) resulted in 107.2 kDa for A2scFv-Fc2 and 108.1 kDa for A2scFv-Fc2DM.

### Example 2: Antigen Specificity

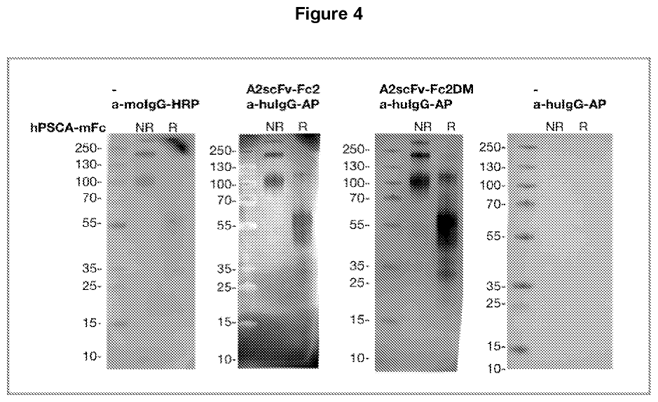

The purified scFv-Fc2 fusion portions were used to detect recombinant human PSCA-mouseFc fusion protein (hPSCA-mFc) by immunoblot (FIG. 4). Both A2scFv-Fc2 and A2scFv-Fc2DM bound to reduced and non-reduced hPSCA-mFc while there was no cross-reactivity of the secondary detection antibody (anti-human IgG-AP) to the mouse Fc of hPSCA-mFc, confirming successful re-engineering and retained antigen specificity of the anti-PSCA scFv-Fc constructs.

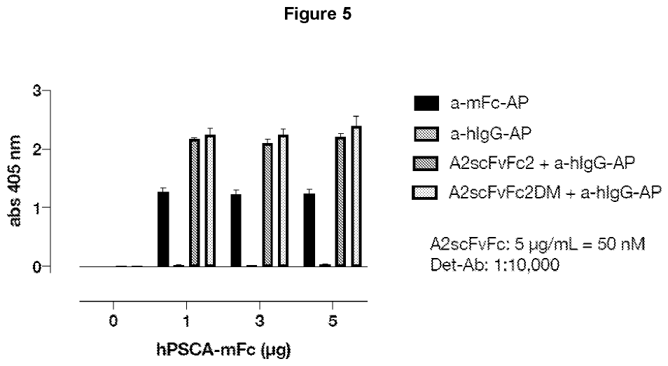

For further confirmation of specific antigen binding ELISA was used to detect binding of A2scFv-Fc2 and A2scFv-Fc2DM to immobilized antigen hPSCA-mFc (FIG. 5). Different concentrations of hPSCA-mFc were coated (1, 3 and 5 μg/mL in PBS, 100 μL/well) overnight, control wells were blocked with BSA. Presence of immobilized antigen was detected using goat anti-mouseFc-AP. Bound anti-PSCA scFv-Fc's were detected using goat anti-human IgGFc-AP antibody and showed increased signal. No nonspecific binding to BSA coated wells was detected.

### Example 3: Antigen Binding Affinity

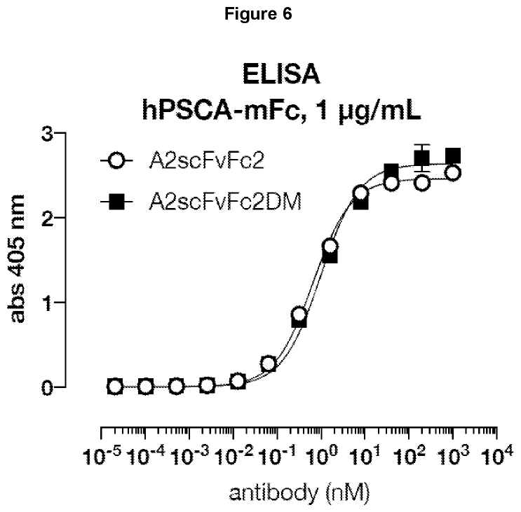

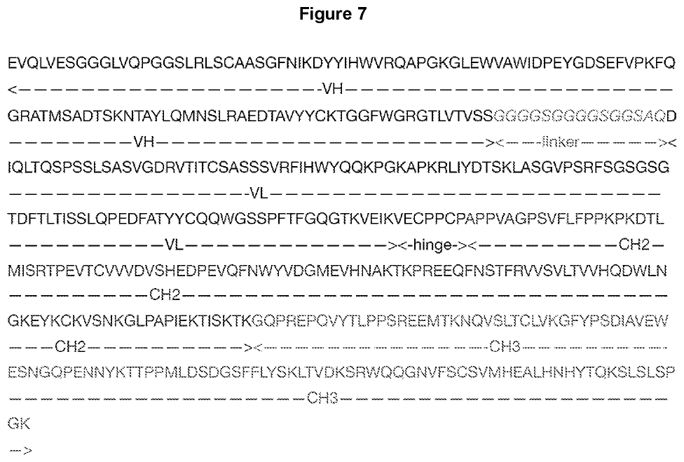

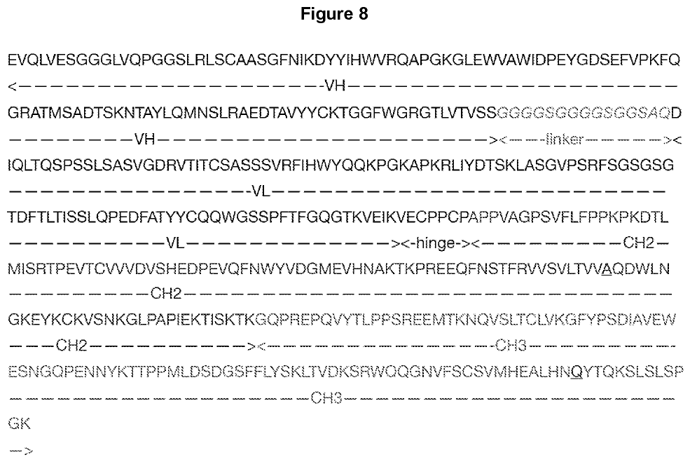

Saturation binding of the anti-PSCA scFv-Fc fragments was tested in ELISA in 3 independent experiments with duplicates. Binding of serial dilutions (1 μM-0.1 μM) of A2scFv-Fc2 and A2scFv-Fc2DM to immobilized recombinant hPSCA-m Fc (1 μg/mL) was specific and dose-dependent (FIG. 6). Half-maximal binding (Kd) was reached at similar concentrations for both constructs (0.5±0.1 nM for A2scFv-Fc2 and 0.8±0.2 nM for A2scFv-Fc2DM). This is an about 4-8-fold improvement in affinity compared to the cys-diabody fragment A2cDb (KD 2 nM on immobilized hPSCA-mFc, QCM and 4 nM on PSCA-expressing cells, flow cytometry) and an approximately 20-30-fold improvement over the A11 Mb (Kd 4 nM on immobilized hPSCA-mFc, QCM and 14 nM on PSCA-expressing cells, flow cytometry).

### Example 4: Cell Binding

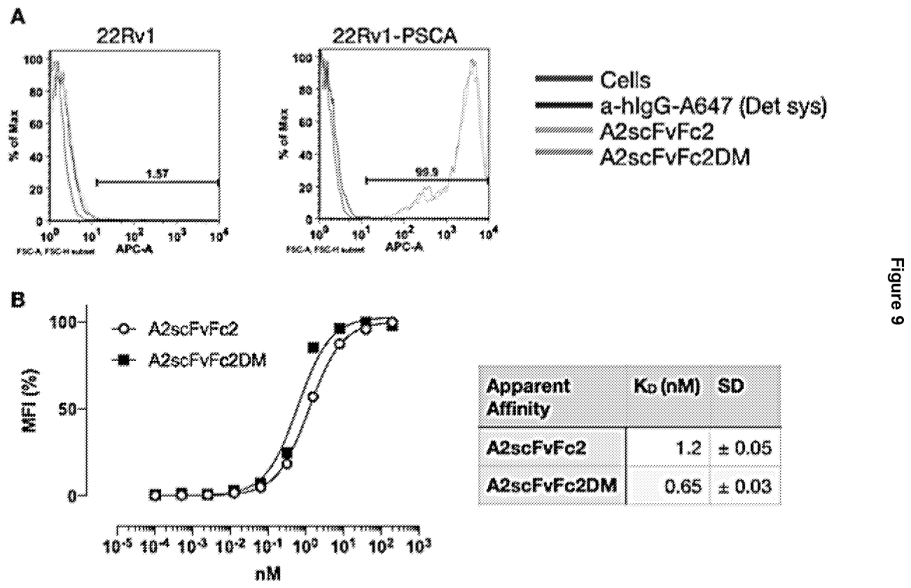

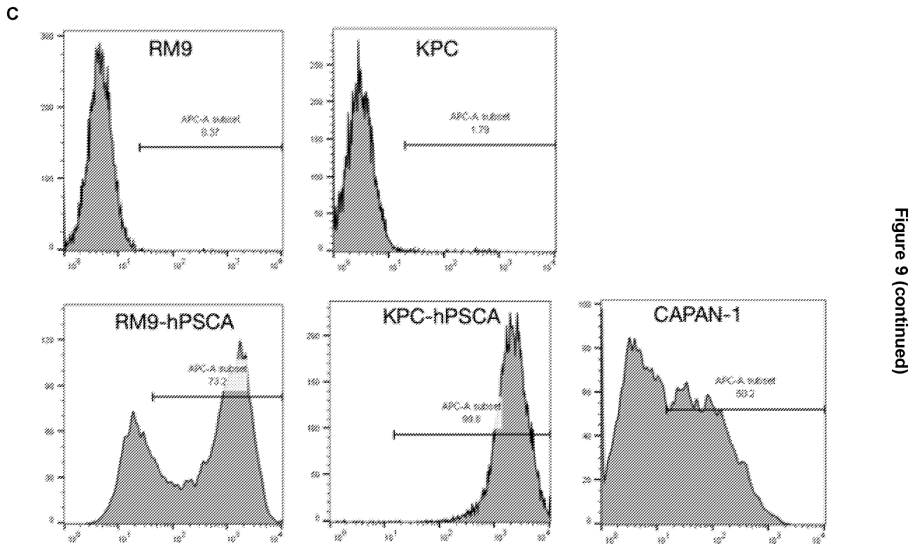

The capability of the anti-PSCA antibody fragments to bind to PSCA expressed on the cell surface was analyzed by flow cytometry. Antigen specificity was confirmed using PSCA-positive and negative cell lines. In this assay binding was only seen with 22Rv1-PSCA cells but not with 22Rv1 (FIG. 9A). Titration of antibody concentration while keeping the cell number constant showed comparable binding of the two A2scFv-Fc variants, with apparent affinities of 1.2±0.05 nM for A2scFv-Fc2 and 0.65±0.03 nM for A2scFv-Fc2DM (FIG. 9B). Binding to murine cell lines transduced to express human PSCA and the human pancreatic cancer cell line Capan-1 was also confirmed using the A2scFv-Fc2 (FIG. 9C).

### Example 5: Radiolabeling

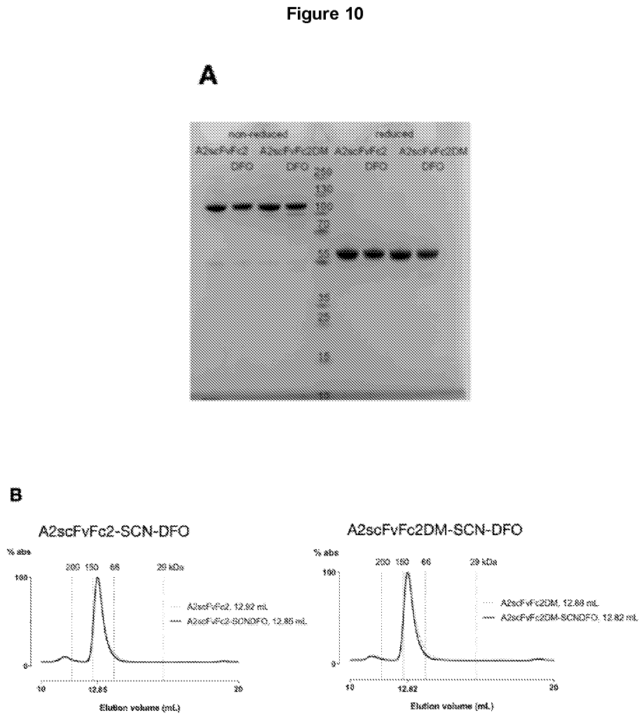

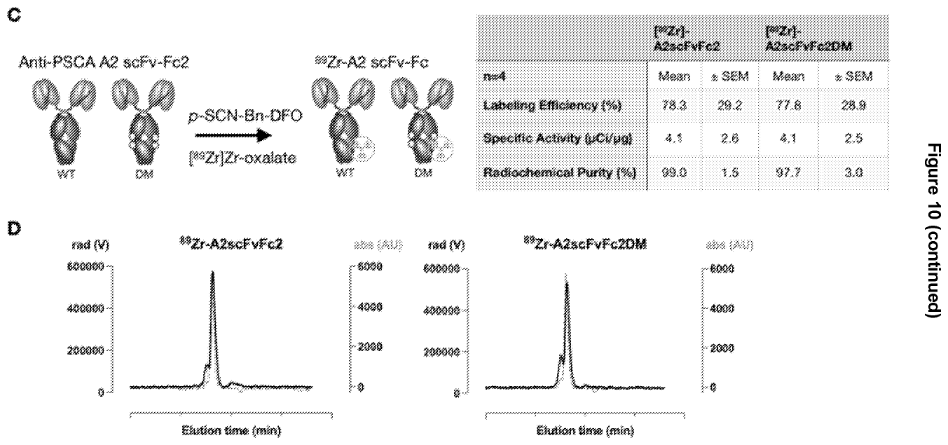

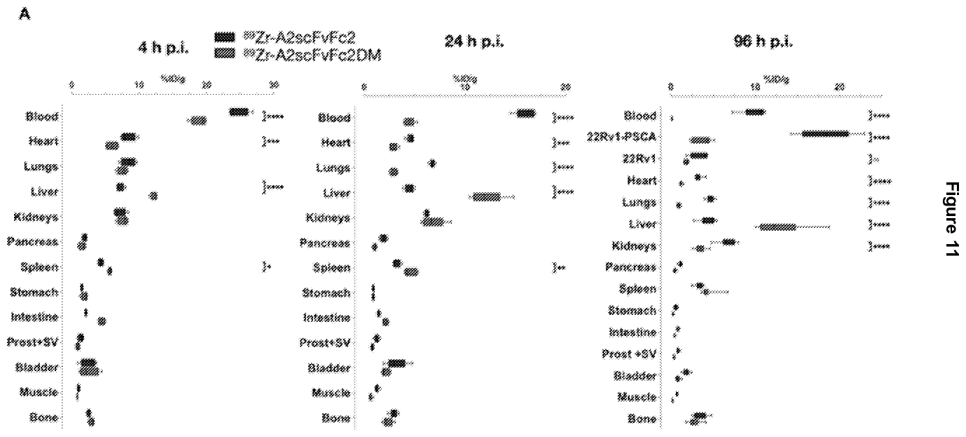

The anti-PSCA A2scFv-Fc2/DM antibody fragments were conjugated with the bifunctional chelator p-SCN-Bn-deferoxamine (DFO) producing thiourea linkages with the amino group of surface-exposed lysine residues. Successful DFO-conjugation was confirmed by SDS-PAGE analysis (FIG. 10A). Size exclusion chromatography analysis of DFO-conjugated antibody fragments revealed a single peak with slightly earlier elution time (compared with the unconjugated fragments), corresponding to a molecular weight shift of approximately 3 kDa which would suggest a chelator:antibody ratio of 4:1. DFO-conjugated antibody fragments were radiolabeled by chelation of ⁸⁹Zr with similar results for labeling efficiencies (around 80%), resulting in specific activities of 4.1±2.5 μCi/μg and yielding radiochemical purities of >95% (FIG. 10C). SEC of the radiolabeled antibody fragments showed overlapping elution profiles for radioactivity and protein (FIG. 10D) confirming that ⁸⁹Zr was chelated/bound to the antibody fragments and no free ⁸⁹Zr remained after purification.

### Example 6: Ex Vivo Biodistribution

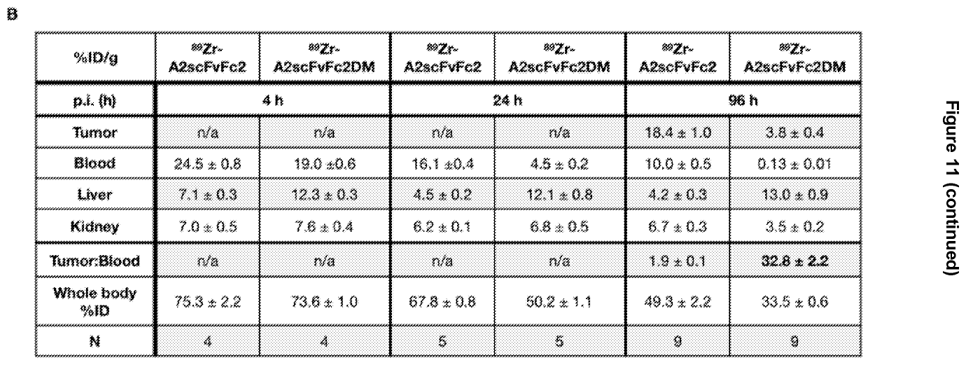

Male nude m ice were injected (via the tail vein) with 10 μg of ⁸⁹Zr-A2scFv-Fc2 or ⁸⁹Zr-A2scFv-Fc2DM, respectively, and groups were euthanized at 4, 24, or 96 hours p.i (FIGS. 11A and 11B). The ex vivo biodistribution data confirm a significant lower blood retention time for ⁸⁹Zr-A2scFv-Fc2DM compared to ⁸⁹Zr-A2scFv-Fc2 (1.3-fold lower at 4 hours p.i. and 3.6-fold lower at 24 hours p.i.) that became more pronounced at later time points (77-fold lower at 96 hours p.i.). This was caused by the mutations impacting FcRn recycling, and the activity was higher for ⁸⁹Zr-A2scFv-Fc2DM in tissues contributing most to antibody recycling such as liver and spleen. Relatively low uptake values in the kidney confirm the shift to hepatic clearance of the scFvFc antibody fragments.

PSCA-specific tumor uptake was confirmed by higher uptake in 22Rv1-PSCA subcutaneous tumors compared to PSCA-negative 22Rv1 tumors. The longer half-life of ⁸⁹Zr-A2scFv-Fc2 resulted in higher accumulation in 22Rv1-PSCA tumors (18.4±1.0% ID/g, 96 hours p.i.), but a lower tumor:blood ratio (1.9±0.1). The ⁸⁹Zr-A2scFv-Fc2DM resulted in a significantly higher tumor:blood ratio (32.8±2.2). The shorter half-life and hepatobiliary clearance and excretion also resulted in an overall smaller fraction of the injected activity retained in the mice over time (whole body % ID was 33.5±0.6% for ⁸⁹Zr-A2scFv-Fc2DM compared with 49.3±2.2% for ⁸⁹Zr-A2scFv-Fc2 at 96 hours p.i.).

Taken together, these results suggest that the double mutant A2scFv-Fc2DM has the potential to improve the RIT therapeutic index, allowing administration of higher activity with reduced hematological and renal toxicity.

### Example 7: ImmunoPET in Nude Mice Bearing Human Prostate Cancer Xenografts (22Rv1-PSCA)

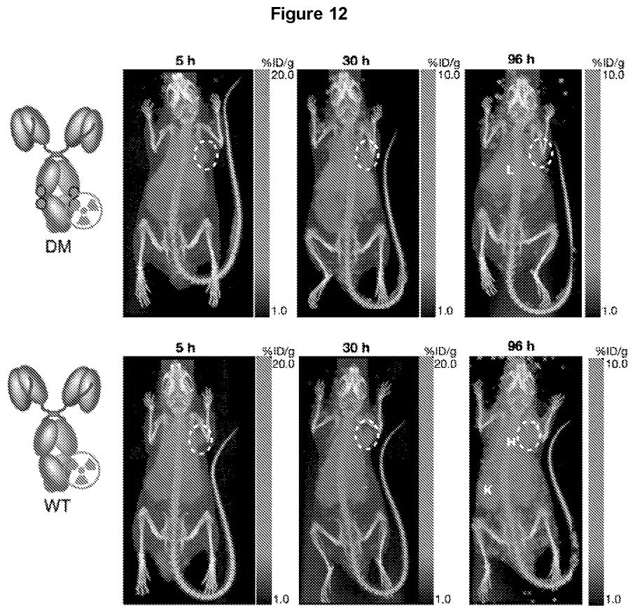

⁸⁹Zr-A2scFv-Fc2 and ⁸⁹Zr-A2scFv-Fc2DM (10 μg/35-70 μCi) were injected intravenously to male nude mice bearing 22Rv1-PSCA xenografts (right shoulder) and static 10-minute PET scans were acquired at 5, 30 and 96 hours p.i. Representative images are shown in FIG. 12. Antigen-specific uptake was observed for both antibody fragments in the PSCA-expressing tumors. The accumulation of activity in the liver of mice injected with ⁸⁹Zr-A2scFv-Fc2DM confirmed the rapid hepatic clearance of the double mutant. High retained activity in the heart on mice injected with ⁸⁹Zr-A2scFv-Fc reflects the longer blood retention time of this tracer.

### Example 8: ImmunoPET in Human PSCA Knock in Mice (hPSCA KI) Bearing Syngeneic Prostate Cancer Xenografts (RM9-PSCA)

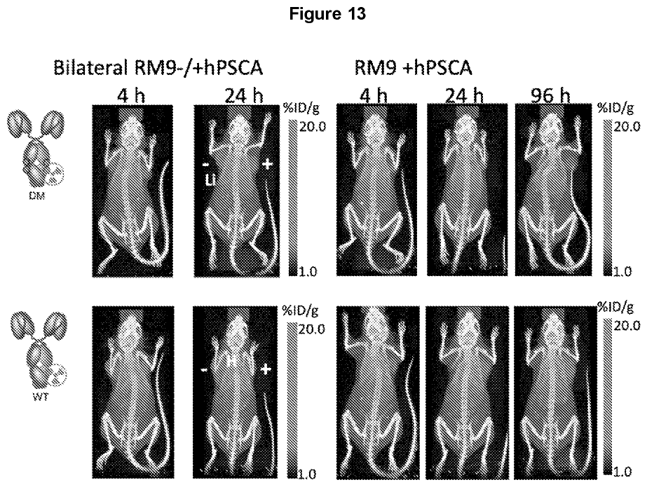

The human PSCA knock in mouse model represents a physiologically more relevant disease model because it enables the evaluation of anti-PSCA antibodies in immunocompetent mice and in the context of normal tissue expression of PSCA. hPSCA KI mice express PSCA at low level in the normal prostate, bladder and stomach, reproducing the expression pattern observed in humans. ImmunoPET studies in hPSCA KI mice bearing bilateral RM9 and RM9-PSCA tumors confirmed the in vivo specificity of both anti-PSCA scFv-Fc antibody fragments, showing higher uptake in RM9-PSCA tumors compared to antigen negative RM9 tumors (FIG. 13). The biodistribution and clearance of ⁸⁹Zr-A2scFv-Fc2 and ⁸⁹Zr-A2scFv-Fc2DM was not altered by the normal tissue expression of PSCA and no increased uptake was observed in bladder or stomach.

### Example 9: Ex Vivo Biodistribution of ⁸⁹Zr-A2scFv-Fc2 and ⁸⁹Zr-A2scFv-Fc2DM in Syngeneic RM9-PSCA in hPSCA KI Mice

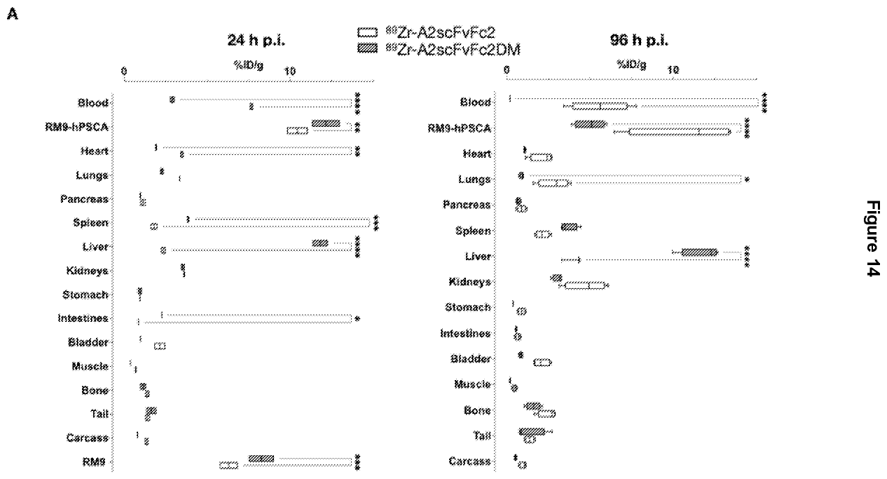

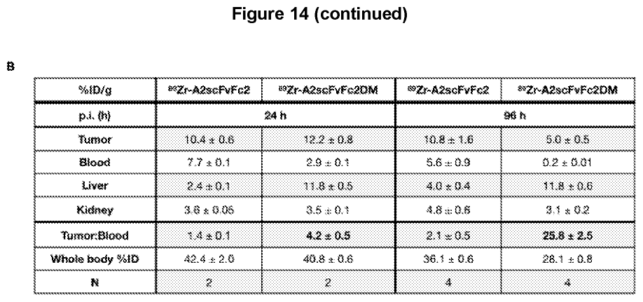

After the last immunoPET scan, mice were euthanized, tissues harvested and analyzed by gamma counting (FIG. 14A). Similar to the xenograft model in nude mice, ⁸⁹Zr-A2scFv-Fc2DM cleared significantly faster from blood (2.9±0.1 vs 7.7±0.1% ID/g at 24 hours p.i. and 0.2±0.01 vs 5.6±0.9% ID/g at 96 hours p.i.). ⁸⁹Zr-A2scFv-Fc2 reached higher uptake in the PSCA positive tumor, but the long circulation time resulted in lower tumor:blood ratios (2.1±0.5 at 96 h) while the tumor:blood ratio for ⁸⁹Zr-A2scFv-Fc2DM reached 25.8±2.5 by 96 hours p.i. The normal tissue expression of PSCA did not lead to higher background uptake and retained whole body activity was comparable to the xenograft model with a lower fraction of the injected dose retained for ⁸⁹Zr-A2scFv-Fc2DM (28.1±0.8% ID) (FIG. 14B).

### Example 10: Plasma Half-Life

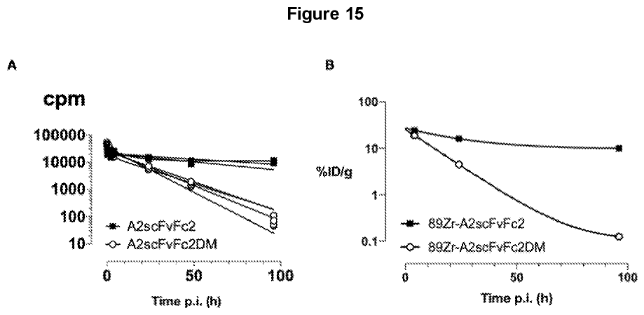

Half-life of ⁸⁹Zr-A2scFv-Fc2 and ⁸⁹Zr-A2scFv-Fc2DM was determined after a single dose i.v. injection into male nude mice bearing bilateral 22Rv1 (71±33 mg) and 22Rv1-PSCA (87±52 mg) tumors. Concentration of the antibodies over a period of 4 days were measured by gamma counting and fitted using a two-phase decay non-linear fit (FIG. 15A). ⁸⁹Zr-A2scFv-Fc2 showed a terminal half-life (t_{1/2β}) of 74.7 hours, while the double mutant ⁸⁹Zr-A2scFv-Fc2DM was eliminated from the blood more rapidly, resulting in a terminal half-life (t_{1/2β}) of 12.2 hours (n=5).

The blood values from the ex vivo biodistribution (FIG. 11) were also used to calculate the terminal half-life and resulted in comparable values: t_{1/2β}=72.77 hours for ⁸⁹Zr-A2scFv-Fc2 and 9.6 hours for ⁸⁹Zr-A2scFv-Fc2DM (FIG. 15B).

1. A genetically engineered anti-prostate stem cell antigen (PSCA) scFv-Fc fusion protein comprising two peptides which form a homodimer, wherein each peptide comprises variable domains VH and VL of an anti-PSCA antibody, and a truncated hinge and fragment crystallizable (Fc) region, and wherein the variable domains are arranged in the order of VH-VL.

2. The fusion protein of claim 1, wherein the variable domains VH and VL are connected by a glycine-rich linker

3. The fusion protein of claim 2, wherein the linker is about 10-25 amino acids.

4. The fusion protein of claim 2 or claim 3, wherein the linker has a sequence of ((G₄S)₂-GGSAQ) (SEQ ID NO: 1).

5. The fusion protein of any one of claims 1-4, wherein the FcRn binding region of the Fc region contains two point mutations H310A and H435Q.

6. The fusion protein of any one of claims 1-5, wherein the truncated hinge and Fc region is derived from human IgG2.

7. The fusion protein of any one of claims 1-6, wherein each peptide of the scFv-Fc fusion protein has an amino acid sequence at least 80%, at least 85%, at least 90%, at least 95%, at least 98%, or at least 99% identical to SEQ ID NO: 2 or SEQ ID NO: 3.

8. The fusion protein of any one of claims 1-7, wherein the scFv-Fc fusion protein is conjugated to an effector moiety.

9. The fusion protein of claim 8, wherein the effector moiety includes a labeling moiety and/or a therapeutic moiety.

10. The fusion protein of claim 9, wherein the labeling moiety comprises one or more radioactive labels or fluorescent labels.

11. The fusion protein of claim 9, wherein the therapeutic moiety includes a cytotoxic agent, an anti-tumor drug, a toxin, a radioactive agent, a cytokine, a second protein, an antibody, a radionuclide, or an enzyme.

12. A pharmaceutical composition comprising an effective amount of one or more scFv-Fc fusion proteins, or scFv-Fc fusion protein and effector moiety conjugates of any one of claims 1-11.

13. The pharmaceutical composition of claim 12, further comprising one or more pharmaceutically acceptable carriers, excipients, and/or stabilizers.

14. The pharmaceutical composition of claim 12 or claim 13, further comprising one or more additional therapeutic agents.

15. The pharmaceutical composition of claim 14, wherein the one or more additional therapeutic agents are conjugated to the scFv-Fc fusion protein.

16. The pharmaceutical composition of claim 14 or claim 15, wherein the additional therapeutic agents include chemotherapeutic agents, cytotoxic agents, cytokines, growth inhibitory agents, and anti-hormonal agents.

17. The pharmaceutical composition of any one of claims 12-16, wherein the pharmaceutical composition is formulated suitable for intravenous, intramuscular, intra peritoneal, intracerobrospinal, subcutaneous, intraarticular, intrasynovial, intrathecal, oral, topical, or inhalation administration.

18. A method of treating or preventing a cancer expressing PSCA comprising administering to a subject an effective amount of one or more scFv-Fc fusion proteins, scFv-Fc fusion protein and effector moiety conjugates, or pharmaceutical compositions of any one of claims 1-17.

19. The method of claim 18, further comprising administering to the subject one or more additional therapeutic agents.

20. The method of claim 19, wherein the additional therapeutic agents include chemotherapeutic agents, radiotherapy, cytotoxic agents, cytokines, growth inhibitory agents, and anti-hormonal agents.

21. A method of detecting cancer expressing PSCA in a subject, comprising:
administering one or more scFv-Fc fusion proteins of any one of claims 1-11 to a subject;
measuring the level of the scFv-Fc fusion proteins in the subject; and
comparing the level of the scFv-Fc fusion proteins with that of a healthy subject or with an average level of a healthy population,
wherein an elevated level of the scFv-Fc fusion proteins in the subject indicating the presence of cancer.

22. The method of claim 21, wherein the scFv-Fc fusion protein is conjugated with a labeling moiety.

23. The method of claim 22, wherein the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable.

24. A method of determining the prognosis of treating a cancer expressing PSCA in a subject, comprising:
administering one or more scFv-Fc fusion proteins of any one of claims 1-12 to a subject;
measuring the level of the scFv-Fc fusion proteins in the subject; and
comparing the level of the scFv-Fc fusion proteins before and after the subject receives a cancer therapy,
wherein a decreased level of the scFv-Fc fusion proteins in the subject after receiving the cancer therapy indicating that the cancer therapy is effective.

25. The method of claim 24, wherein the scFv-Fc fusion protein is conjugated with a labeling moiety.

26. The method of claim 25, wherein the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable.

27. A method of imaging a cancer expressing PSCA in a subject, comprising:
administering one or more scFv-Fc fusion proteins of any one of claims 1-12 to a subject; and
imaging the subject to determine the location and size of the tumor.

28. The method of claim 27, wherein the scFv-Fc fusion protein is conjugated with a labeling moiety.

29. The method of claim 28, wherein the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable.

30. The method of any one of claims 18-29, wherein the cancer expressing PSCA includes prostate cancer, pancreatic cancer, and bladder cancer.


SEQUENCE LISTING (5 sequences)

SEQ ID NO: 1 - protein, 7 aa, Artificial Sequence
  linker
GSGGSAQ

SEQ ID NO: 2 - protein, 456 aa, Artificial Sequence
  scFv-Fc fusion protein
  MISC_FEATURE (1)..(112): VH
  MISC_FEATURE (113)..(127): linker
  MISC_FEATURE (128)..(233): VL
  MISC_FEATURE (234)..(240): hinge
  MISC_FEATURE (241)..(349): CH2
  MISC_FEATURE (350)..(456): CH3
EVQLVESGGGLVQPGGSLRLSCAASGFNIKDYYIHWVRQAPGKGLEWVAWIDPEYGDSEFVPKFQGRATMSADTSKNTAYLQMNSLRAEDTAVYYCKTGGFWGRGTLVTVSSGGGGSGGGGSGGSAQDIQLTQSPSSLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 456 aa, Artificial Sequence
  scFv-Fc fusion protein
  MISC_FEATURE (1)..(112): VH
  MISC_FEATURE (113)..(127): linker
  MISC_FEATURE (128)..(233): VL
  MISC_FEATURE (234)..(240): hinge
  MISC_FEATURE (241)..(349): CH2
  MISC_FEATURE (350)..(456): CH3
EVQLVESGGGLVQPGGSLRLSCAASGFNIKDYYIHWVRQAPGKGLEWVAWIDPEYGDSEFVPKFQGRATMSADTSKNTAYLQMNSLRAEDTAVYYCKTGGFWGRGTLVTVSSGGGGSGGGGSGGSAQDIQLTQSPSSLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKVECPPCPAPPVAGPSVFLFPPKPKDTLMISRTPEVTCVVVDVSHEDPEVQFNWYVDGMEVHNAKTKPREEQFNSTFRVVSVLTVVAQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNQYTQKSLSLSPGK

SEQ ID NO: 4 - protein, 114 aa, Homo sapiens
  MISC_FEATURE (1)..(11): signal peptide
  MISC_FEATURE (86)..(114): propeptide
MAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 5 - protein, 20 aa, Homo sapiens
  MISC_FEATURE (1)..(5): truncated residues
  MISC_FEATURE (8)..(8): disulfide bridge
  MISC_FEATURE (11)..(11): disulfide bridge
ERKCCVECPPCPAPPVAGPS